(19) United States
(12) Patent Application Publication
(10) Pub. No.: US 2012/0214836 A1
(43) Pub. Date: Aug. 23, 2012

(54) SENSITIZER, PHARMACEUTICAL COMPOSITION, KIT AND USE FOR TARGET THERAPY

(76) Inventor: Pei-Ru Liao, Taipei City (TW)

(21) Appl. No.: 13/238,649

(22) Filed: Sep. 21, 2011

(30) Foreign Application Priority Data
Feb. 23, 2011 (TW) 100106076

(51) Int. Cl. A61K 31/436; A61P 35/00

(52) U.S. Cl. 514/291

(57) ABSTRACT

A target therapy sensitizer including rapamycin and substituted quinoline is disclosed. In addition, a pharmaceutical composition containing the sensitizer, a target therapy sensitization kit containing the two aforementioned compounds, and a use of a combination of the two aforementioned compounds as a target therapy sensitizer are also disclosed. The application of the target therapy sensitizer, pharmaceutical composition, kit and use is advantageous for improving the treatment effect of target therapies.

### CROSS REFERENCE TO RELATED APPLICATIONS

This Non-provisional application claims priority under 35 U.S.C. §119(a) on Patent Application No(s). 100106076 filed in Taiwan, Republic of China on Feb. 23, 2011, the entire contents of which are hereby incorporated by reference.

### BACKGROUND OF THE INVENTION

1. Field of Invention

The present invention relates to a sensitizer, a pharmaceutical composition, a kit and use. More particularly, the present invention relates to a sensitizer, a pharmaceutical composition, a kit and use for target therapy.

2. Related Art

Rapamycin, also known as sirolimus, was isolated from Streptomyces hygroscopicus of the soil sample obtained from Easter Island. Rapamycin has demonstrated anti-fungal activity both in vitro and in vivo, especially in Candida albicans, and thus is used as an agricultural antifungal agent in the early days. However, when the fact that rapamycin has an inhibition effect against immunoreaction and anti-proliferative effect has been discovered, rapamycin is used on large scale as an immuno-inhibition drug, to inhibit the rejection of, for example, acute allotransplantation.

There are a variety of commercial drugs containing rapamycin now, for example, RAPAMUNE® (Wyeth, Collegeville, Pa., USA), the main use of which is for preventing organ rejection after kidney transplant. In addition, CERTICAN® produced by Novartis, East Hanover, N.J., USA, also provides the effect of preventing organ rejection.

On the other hand, chloroquine is a substituted quinoline which has been commonly used for over 60 years for the prevention and treatment of malaria prophylaxis, or for rheumatoid arthritis treatment and HIV treatment.

It has been testified that rapamycin and chloroquine has certain degree of treatment effect on a cancer and a tumor due to their property of inhibiting cell growth and inducing cell death. Relative research is also revealed in various international journal articles. In addition, the technique of combining rapamycin and chloroquine to inhibit the growth and proliferation of tumor has been disclosed in both US patent 2006/2064384 and WO patent 2010/132233.

However, the follow-up studies did not make any further breakthrough, and comparing to using individually, the use of their combination does not show obviously promoted effects. Moreover, when using in certain type of cancers, the treatment effects of using rapamycin, chloroquine and their combination are almost the same and show no advantage of combination use. On the other hand, there has been no report or article of using a combination of rapamycin and substituted quinoline as a sensitizer of cancer treatment, instead of a therapeutically active composition.

### SUMMARY OF THE INVENTION

In view of the deficiency of prior art, the present invention is developed. An objective of the present invention is to provide a target therapy sensitizer, a pharmaceutical composition, a kit and use, which can substantially improve the treatment effect of the active composition in a target therapy by using the combination of rapamycin and substituted quinoline.

A target therapy sensitizer according to the present invention includes rapamycin and substituted quinoline. Preferably, the target therapy sensitizer comprises an effective amount of rapamycin and substituted quinoline, a pharmaceutically acceptable carrier, diluent, excipient or a combination thereof.

A pharmaceutical composition according to the present invention includes the aforementioned target therapy sensitizer, an effective amount of therapeutically active composition, and a pharmaceutically acceptable carrier, diluent, excipient or a combination thereof.

A target therapy sensitizer kit according to the present invention includes rapamycin and a first pharmaceutically acceptable carrier, diluent, excipient, or a combination thereof; and substituted quinoline and a second pharmaceutically acceptable carrier, diluent, excipient, or a combination thereof.

A use of a combination of a rapamycin and a substituted quinoline according to the present invention is as a target therapy sensitizer.

In order to demonstrate the technical features of the present invention in the follow-up contents, specific terms are defined hereunder, and the detail of the present invention will be illustrated thereafter. In addition, the target therapy sensitizer comprises both rapamycin and substituted quinoline, therefore, when a combination of rapamycin and quinoline is mentioned in the specification, it is considered to include the target therapy sensitizer and the product of the preparation of a target therapy sensitizer kit.

As used herein, a “target therapy sensitizer” refers to a composition containing at least two materials. Preferably, it refers to a composition containing an effective amount of two materials and a pharmaceutically acceptable carrier, diluent, excipient or a combination thereof. For example, the aforementioned composition can be applied before, during or both before and during the target therapy to improve or enhance the effect of one or more effective amount of therapeutically active composition upon a cancer or a tumor in an individual in need, and then achieve the goal of eliminating, inhibiting, improving, comforting or preventing a cancer and its symptoms; retarding, prohibiting, reversing the rate of tumor proliferation; or the medical effects similar to the foregoing goals. Wherein the composition mentioned above can be a combination of rapamycin and substituted quinoline.

To continue, “to improve or enhance the effect of a therapeutically active composition upon a cancer or a tumor in an individual in need” means to improve, enhance or intensify the cytotoxicity of the therapeutically active composition to a cancer, a cancer cell, a variant cell, a tumor cell or a combination thereof, to decrease the resistance of a cancer cell or various type of the abovementioned cells to the therapeutically active composition, to induce apoptosis of a cancer cell or various type of the abovementioned cells initiated by the therapeutically active composition, or the combination of the above, by using the aforementioned composition through, for example, specific biochemical reactions or signal transduction pathways (like phosphorylation of a specific protein).

[Rapamycin]

As used herein, “rapamycin” refers to one selected from a group of compounds containing basic rapamycin structure (Formula I), including the derivatives obtained from chemical/biological modification or substitution, while still maintaining the nature of the original basic rapamycin structure or possessing similar properties with that of the original basic rapamycin structure. Therefore, “rapamycin” substantially comprises easters, ethers, enzymes, hydrazones, hydroxylamines, and rapamycins with the functional groups of the basic rapamycin structure modified by reduction, oxidation or substitution. Needless to say, “rapamycin” also includes pharmaceutically acceptable salts formed from the acidic/basic bases of Formula I.

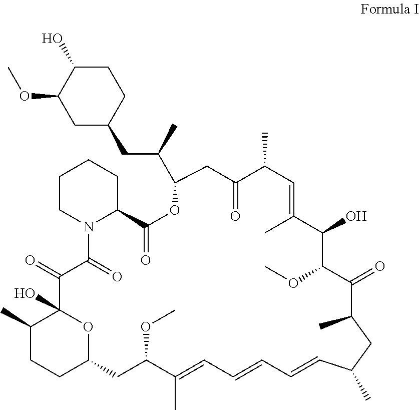

Rapamycin is referred to sirolimus, which is well-known in the present field, and it also comprises other compounds with identical or similar structures but different trade names. Further, except rapamycin, it is also proper for the present invention to use a compound selected from the group consisting of everlimus, temsirolimus, tacrolimus, 2-(dimethyl phosphinyl)sirolimus (deforolimus), biolimus and (42S)-42-deoxy-42-(1H-tetrazol-1-yl)-rapamycin (zotarolimus).

[Substituted Quinoline]

As used herein, “substituted quinoline” not only refers to chloroquine, but also includes, but not confines to, derivatives or chloroquine homologue obtained by further modification or substitution of chloroquine. Among which, derivatives obtained by further modification or substitution of chloroquine can possess the chemical structure of chloroquine, but one or more hydrogens or functional groups thereof are modified or substituted by one or more substituents. Specifically, the substituents mentioned above can be halogen, C₁₋₁₀ alkyl, —OC₁₋₁₀ alkyl, hydroxyl, C₆₋₁₀ aryl, heteroaryl, heterocycloalkyl, alkheterocycloalkyl, heteroalkyl or alkheteroalkyl. For example, substituted quinoline can be selected from a group consisting of free hydroxychloroquine, primaquine, amoproquine, amodiaquine, cycloquine, sontoquine, quinacrine, tebuquine and bis-pyroquine.

[Compound]

All of the compounds or materials, which are mentioned herein and substantially related to the technical feature of present invention, include the compounds or materials themselves and any pharmaceutically acceptable forms thereof. Among which, the pharmaceutically acceptable forms can be exemplified, but not limited to, various isomers including diastereomers and enantiomers, salts, ionization forms, solvent, prodrug, polymorph and racemic mixture.

[Ratio and Usage]

For the combination of rapamycin and substituted quinoline to perform sensitizing effects, the weight percentage ratio of an effective amount of rapamycin and substituted quinoline can be between about 1:6×10⁸ to about 2000:1. However, the sensitizing effect of different ratio within the scope can be greatly different, thus, the advisable ratio of application is between about 1:100 to about 1:5000, preferably is between about 1:1000 to about 1:3000, more preferably is about 1:2000. Needless to say, said ratio can also be applied to the pharmaceutical composition of the present invention, or can be used as the preparation ratio of the target therapy sensitizer of the present invention when it is used in practice.

An example according to the ratio stated above is described hereinbelow. When the combination of rapamycin and substituted quinoline are prepared as a solution form for oral administration or injection, the effective amount of rapamycin may range from about 1 pg/ml to about 1 μg/ml, and the effective amount of substituted quinoline may range from about 0.5 ng/ml to about 0.6 mg/ml. Preferably, the effective amount of rapamycin may range from about 100 pg/ml to about 10 ng/ml, and the effective amount of substituted quinoline may range from about 10 ng/ml to about 15 μg/ml.

The combination of rapamycin and substituted quinoline according to the present invention can be administrated together with an effective amount of therapeutically active composition to an individual in need. Among which, as used herein, “an effective amount” refers to an amount of the materials or the compounds mentioned, which can effectively inhibit or treat the syndromes of a cancer or retard or reverse the rate of tumor proliferation.

As used herein, “a therapeutically active composition” refers to one or more natural material or a product obtained from processing a nature material, or a synthesized compound, wherein within the effective dose range, it can perform treatment or prevention effects on specific diseases, adjustment disorders or indicates by changing the physiological condition of the subject received administration. For example, the therapeutically active composition can be a compound, preferably a small molecular composite, an antibody, an antibody-drug complex or a combination thereof. Specifically, a therapeutically active composition can be, for example, Erlotinib, Herceptin, Gefitinib, etc.

In particular, as used herein, “administration together” refers to administrating the combination of rapamycin and substituted quinoline before, during or both before and during the application of the therapeutically active composition. Specifically, for example, to administrate with the therapeutically active composition at the same time within the same treating procedure, to separately administrate at different but close points of time within a day, or even to administrate individually before the application of the therapeutically active composition in a different day with its effect maintained.

As used herein, “an individual in need” refers to an animal suffering from a cancer, having a tumor formed in its body, having the symptoms of a cancer or a tumor, having a tendency of suffering from a cancer, or having a tumor formed inside of its body. Said animal includes mammals and preferably refers to humans.

After administrating to an individual in need, the rapamycin and the substituted quinoline cause a synergistic effect, and therefore performs a further remarkable sensitizing effect comparing to when the rapamycin or the substituted quinoline is used individually. In addition, although rapamycin and substituted quinoline have been used as therapeutically active composition in the prior art, i.e. an anti-cancer or anti-tumor drug, but the treatment effects of both are quite limited. The applicant discovers that the combination of rapamycin and substituted quinoline is not suitable for acting as an active composition providing treatment effects in a treatment procedure, but should be playing an assisting part therein, which is, the target therapy sensitizer disclosed in the present invention.

[Treating Subjects and Administration Pathways]

The combination of rapamycin and substituted quinoline according to the present invention can be applied in any form of target therapies of a cancer or a tumor, and is especially suitable for the application on, for example, lung cancer, colon cancer or breast cancer; particularly suitable for all kinds of solid cancers, to treat a tumor, retard the growth of a tumor or prevent the formation of a tumor.

The combination of rapamycin and substituted quinoline of the present invention can be formed in, for example but not limited to, a solid or liquid oral administration dosage form, such as a tablet or a capsule, preferably produced together with an effective amount of therapeutically active composition into an oral administrative tablet. Of course, it can also be formed as other available dosage forms, such as pills, sachets, granules, powders, chewing gums, suspensions, emulsions, suppositories and solutions.

Other than oral administration, the combination of rapamycin and substituted quinoline can also be administrated individually or together with the therapeutically active composition via a pathway outside the gastrointestinal tract, for example but not limited to, intravenous injection, or subcutaneous, intramuscular, intrathecal, intraperitoneal, intrarectal, viginal, nasal, intragastric, intratracheal, pulmonary, intratumoral or peritumoral injection or implantation.

To sum up, the target therapy sensitizer, pharmaceutical composition, kit and use of the present invention apply the combination of rapamycin and substituted quinoline to increase the sensitization of a cancer cell or a tumor, thereby when applying in cooperation with a therapeutically active composition, they can achieve the goal of improving or increasing the effect of the therapeutically active composition upon a cancer or a tumor in an individual in need.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is the data map of apoptosis result under the reaction of Gefitinib analyzed by a Cytometer.

FIG. 2 is the data map of apoptosis result under the reaction of Erlobinib analyzed by a Cytometer.

### DETAILED DESCRIPTION OF THE INVENTION

The present invention will be apparent from the following detailed description, which proceeds with reference to the accompanying drawings, wherein the same references relate to the same elements. The ratio and usage of rapamycin, substituted quinoline and the combination thereof can be referred to the paragraphs above and thus will not be re-illustrated here.

A target therapy sensitizer according to the present invention comprises rapamycin and substituted quinoline. In the present embodiment, the rapamycin is a compound of Formula I described above, and the substituted quinoline is chloroquine.

The method of preparing rapamycin and substituted quinoline is well-known by a person having ordinary skill in the art. Further, it is easy to understand the technique of producing a target therapy sensitizer with both rapamycin and substituted quinoline. In the present embodiment, a target sensitizer can further include a pharmaceutically acceptable carrier, diluent, excipient or a combination thereof for preparing a compatible dosage form or prescription form, wherein the pharmaceutically acceptable carrier, diluent or excipient can be, for example, well-known magnesium carbonate, magnesium stearate, talc, sugar, lactose or a combination thereof.

The preparation of a target therapy sensitizer is not limited to uniformly mixing rapamycin and substituted quinoline, that is, in target therapy sensitizers with the same dosage form, rapamycin and substituted quinoline can be mixed in other ratio, or even not being mixed. For example, a target therapy sensitizer can be a tablet or a capsule, wherein part of it being rapamycin and another part of it being substituted quinoline. Preferably, in the present embodiment, rapamycin and chloroquine can be bought from a vendor (please refer to the experimental examples), and a target therapy sensitizer can be powders prepared by mixing powdery rapamycin and substituted quinoline.

Generally speaking, one with ordinary skill in the art can easily know the compatible dosage range of rapamycin and substituted quinoline, which is also the compatible dosage range of the present invention. For example, the acceptable dosage range of rapamycin for an individual in need is between about 0.001 mg to about 1000 mg per kg per day, and the acceptable dose range of substituted quinoline for an individual in need is between about 0.001 mg to about 1000 mg per kg per day. Preferably, the dosage range of rapamycin is between about 0.1 mg to about 100 mg per kg per day, and the dosage range of substituted quinoline is about 1.5 mg to about 150 mg per kg per day. Certainly, it is essential for the dosage of rapamycin or substituted quinoline to alter according to the therapeutically active composition, the administration pathway, or the physiological condition of an individual in need. Generally speaking, oral administration requires higher dosage, but on the other hand, it requires relatively lower dosage during the primary stage of the treatment.

There may be a dosage unit of a target therapy sensitizer in every formulation, that is, one formulation contains enough dosage for performing sensitizing effect in an individual in need, as is convenient for direct administration. In the present embodiment, every package of powders contains a dosage unit of target therapy sensitizer. Needless to say, in other embodiments of the present invention, one dosage unit of a target therapy sensitizer can be separated into several sub-dosage units or sub-packages, for example, separated into two to three tablets or capsules and packed in the same blister pack.

In the present embodiment, because a target therapy sensitizer is separated from an effective amount of therapeutically active composition, the method of individual administration is adopted during application. For example, in a treatment procedure using a therapeutically active composition, the target therapy sensitizer can be applied before, during, or both before and during the application. To be specific, the target therapy sensitizer can be administrated with the therapeutically active composition at the same time or at a different time of the day, for example, with an interval of 1 or 5 hours. Besides, the frequency and order of administration of a target therapy sensitizer is not limited. In the present embodiment, during one treatment procedure, only one administration of a target therapy sensitizer is required. And in other embodiments, a target therapy sensitizer is provided every time before the administration of a therapeutically active composition, or a target therapy sensitizer is re-administrated during the period of every two, three or five times of administration of a therapeutically active. However, the present invention is not limited to this.

Further, in the present embodiment, when a target therapy sensitizer is applied before, during or both before and during application of a target therapy, rapamycin and substituted quinoline cause a synergistic effect, so as to improve or enhance the effect of one or more therapeutically active composition of the amount effective upon a cancer or a tumor in an individual in need, and thus to retard, prohibit or reverse the rate of tumor proliferation, or to achieve the medical effects similar to the foregoing goals. In particular, by using the target therapy sensitizer of the present invention and through specific biochemical reaction pathways, for example, the phosphorylation of a specific protein, it is capable of improving, enhancing or intensifying the cytotoxicity of a therapeutically active composition to a cancer, a cancer cell, a variant cell, a tumor cell or a combination thereof, decreasing the resistance of a cancer cell or various type of the abovementioned cells to the therapeutically active composition, inducing apoptosis of a cancer cell or various type of the abovementioned cells initiated by the therapeutically active composition, or the combination of the three mentioned above.

In another embodiment, the present invention also provides a pharmaceutical composition, wherein is contains a target therapy sensitizer, an effective amount of therapeutically active composition and a pharmaceutically acceptable carrier, diluent, excipient or a combination thereof. Among which, the illustration of the target therapy sensitizer and therapeutically active composition can be referred to in the paragraphs above and thus will not be described here again, but only the insufficient or unclear part of which to be further explained. The therapeutically acceptable carrier, diluent or excipient can be used in proper form of solid or liquid, however, the solid form is preferred, for example, well-known magnesium carbonate, magnesium stearate, talc, sugar, lactose or a combination thereof.

Specifically, in the present embodiment, the pharmaceutical composition can be made into a single dosage, like a tablet or powders, by mixing a target therapy sensitizer and an effective amount of therapeutically active composition at a specific ratio, which is beneficial, for example, for providing the treatment of specific cancers and thus has different orientation from the aforementioned target therapy sensitizer which is made and administrated independently and thus is more flexible. In the present embodiment, the ratio of the target therapy sensitizer and the therapeutically active composition can be from 1000:1 to 0.1:1, preferable 100:1 to 1:1. Of course, said ratio can be adjusted according the subject received administration, its physiological condition and the type of cancer. As for the method of preparing the pharmaceutical composition, the dosage form and the composition besides the target therapy sensitizer can all be completed by one with ordinary skill in the art according to the disclosure of the present invention.

In yet another embodiment, the present invention provides another target therapy sensitizer kit, wherein it is applied with an effective amount of therapeutically active composition to an individual in need. Among which, the target therapy sensitizer kit includes a rapamycin, a first pharmaceutically acceptable carrier, diluent or excipient, a substituted quinoline and a second pharmaceutically acceptable carrier, diluent or excipient, wherein the related description of the rapamycin, the substituted quinoline and the rest of it can be referred to in the paragraphs above and thus will not be re-illustrated here.

The first and the second pharmaceutically acceptable carrier, diluent, excipient or a combination thereof can be a well-known material or composition, and the method and dosage form of preparing a prescription of each of them with rapamycin or substituted quinoline are also prior art in the field of the present invention. In particular, in the present embodiment, there can be an individual pack or container, such as a blister pack, for a target therapy sensitizer kit, to contain or store, for example, the tablet made of rapamycin and a first pharmaceutically acceptable excipient and the tablet made of substituted quinoline and a second pharmaceutically acceptable excipient. Said individual pack can be provided to an individual in need during application or be administrated to an individual in need after preparation.

Needless to say, in other embodiments, the rapamycin and the substituted quinoline can be processed together into an injection form, which is obtained by preparing according to a specific ratio at the time of administration or within a suitable time period before the administration, for example, within one to several weeks (such as within 10 days, 5 days or 24 hours). Accordingly, when rapamycin and substituted quinoline need to be administrated separately, in different dosage forms, or when the compositional ratio of which needs to be adjusted, it is especially beneficial to use the target therapy sensitizer kit of the present invention.

In yet another embodiment, the present invention provides a use of a combination of rapamycin and quinoline as a sensitizer, wherein the related description of the rapamycin, the substituted quinoline and other parts can be referred to in the paragraphs above and thus will not be re-illustrated here.

As mentioned above, according to the target therapy sensitizer, pharmaceutical composition, kit and use of the present invention, a combination of rapamycin and substituted quinoline is used to increase the sensitivity of cancer cells or tumors and therefore achieving the improvement or enhancement of the effect of the therapeutically active composition upon a cancer or a tumor in an individual in need.

### EXPERIMENTAL EXAMPLE 1

### To Prepare the Target Therapy Sensitizer

The rapamycin and chloroquine are purchased individually from Sigma-Aldrich, Inc, St. Louis, Mo., USA. Obtain about 1 mg of rapamycin and about 2 g of substituted chloroquine by a weighting scale in room temperature, mix the powder rapamycin and chloroquine uniformly in a weight percentage ratio of 1:2000. And then, pack it into a dosage form of powders or sachets and store in room temperature.

### EXPERIMENTAL EXAMPLE 2

### Preparation of a Pharmaceutical Composition

Likewise, purchase rapamycin and chloroquine from Sigma-Aldrich, Inc, St. Louis, Mo., USA and produce a target therapy sensitizer according to the method described in Experimental Example 1 there after. And then, obtain about 1 mg of the target therapy sensitizer and about 0.1 mg of Erlotinib and mix them uniformly in a weight percentage ratio of about 10:1, add a sufficient amount of magnesium carbonate as a pharmaceutical acceptable carrier, make it into a pharmaceutical composition in the form of tablets and store in room temperature likewise.

### EXPERIMENTAL EXAMPLE 3

### Using the Target Therapy Sensitizer to Enhance the Effect of Gefitinib Upon the Apoptosis of a Cancer Cell

Culture epidermoid carcinoma cell line A431 by tissue culture technique in DMEM supplemented with 10% FBS to a proper amount. And then, take a suspension containing 1×10⁶ cells and inoculate it in a 6-wells plate. In addition, prepare 10 nM rapamycin, 10 μM chloroquine and 5 μM Gefitinib solution separately in room temperature. Similarly, add the above-mentioned solutions in room temperature in sequence. And then, put the plate into a 37° C. incubator to continue culturing for 48 hours. Remove the broth and chemical in each well in sequence, add PBS to wash, and then use trypsin-EDTA to obtain cells and analyze the apoptosis.

Apoptosis is analyzed by the method of phosphatidylserine externalization using an analysis kit for apoptosis, Annexin V (BD Pharmingen), the procedures are in accordance with the manual. In summary, wash the obtained A431 cells with PBS for 3 times, and use Annexin V/propidium iodide (PI) to dye part of the cells. Add 1% BSA to those cells that have been treated and then add 222.5 μl binding buffer; dye directly with 10 μl PI and 2.5 μl Annexin V-FITC, and immediately move the reaction to an environment with low temperature and no light for 10 minutes. Calculate the percentage of apoptosis by a Flow cytometer and its software, FACSCalibur.

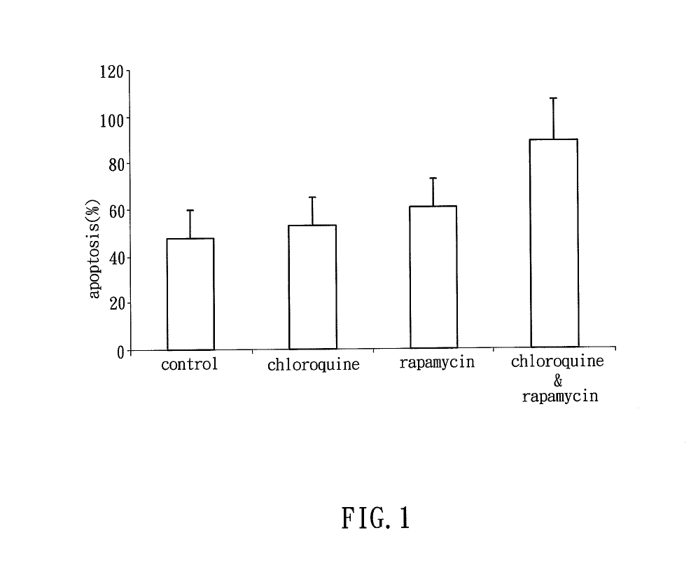

FIG. 1 is the data map of apoptosis under the reaction of Gefitinib analyzed by a Flow cytometer. Referring to FIG. 1, the one marked as “control” is the apoptosis ratio of A431 cells according to the same treatment procedure but with no addition of chloroquine or rapamycin; the ones marked as “chloroquine” or “rapamycin” are individually the apoptosis ratio of A431 cells according to the same treatment procedure with the addition of either chloroquine or rapamycin, and the one marked as “chloroquine and rapamycin” means the addition of both compounds. It is clear from the data that in the target therapy using Gefitinib as the active compound, when chloroquine or rapamycin is used individually as a sensitizer, the apoptosis ratio is raised but not over 15%. However, when chloroquine and rapamycin are used together, apoptosis ratio increases to about 40% to 45%.

### EXPERIMENTAL EXAMPLE 4

### Using the Target Therapy Sensitizer to Enhance the Effect of Erlotinib Upon the Apoptosis of a Cancer Cell

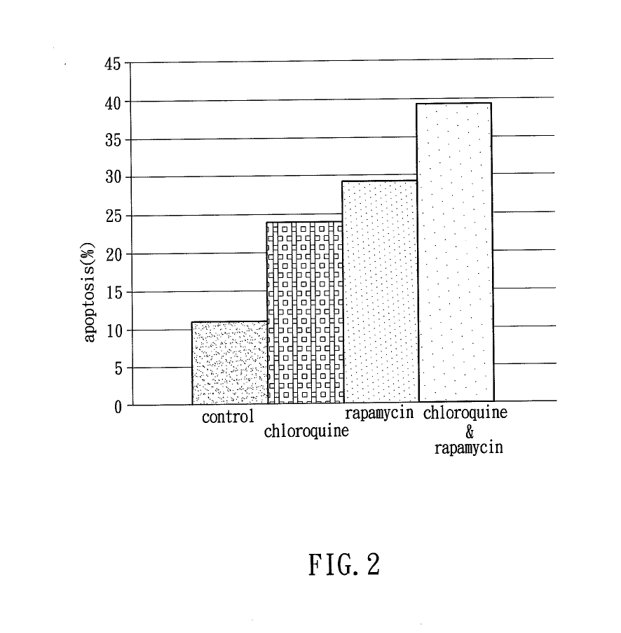

Except for using 0.5 μM Erlotinib as the therapeutically active composition, follow the approximately identical steps and conditions with Experimental Example 3. FIG. 2 is the data map of the result of apoptosis under the reaction of Erlotinib in Experimental Example 4 analyzed by a Flow cytometer. Referring to FIG. 3, it is clear from the data result that the combination of chloroquine and rapamycin remarkably enhanced the effect of Erlobinib upon the apoptosis of A431 cells up to 30%, which is better than the control groups of not using them or using them separately.

What is described above is not limited but only exemplary. Any modification or alteration with the spirit and within the scope of the present invention should be comprised in the Claims stated below.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A target therapy sensitizer, comprising rapamycin and substituted quinoline.

2. The target therapy sensitizer of claim 1, wherein the rapamycin is a compound of the following formula:

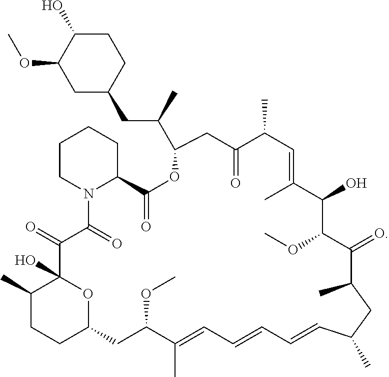

3. The target therapy sensitizer of claim 1, wherein the substituted quinoline is a chloroquine.

4. The target therapy sensitizer of claim 1, wherein the weight percentage ratio of the rapamycin to the substituted quinoline is between 1:100 and 1:5000.

5. The target therapy sensitizer of claim 1, wherein the rapamycin and the substituted quinoline cause a synergistic effect.

6. The target therapy sensitizer of claim 1, which is administrated together with an effective amount of therapeutically active composition to an individual in need.

7. The target therapy sensitizer of claim 6, which is administrated together with the therapeutically active composition to improve or enhance the effect of the therapeutically active composition to a cancer or a tumor in the individual in need.

8. A pharmaceutical composition comprising a target therapy sensitizer, which comprising rapamycin and substituted quinoline, an effective amount of therapeutically active composition, and a pharmaceutically acceptable carrier, diluent, excipient, or a combination thereof.

9. The pharmaceutical composition of claim 8, wherein the rapamycin is a compound of the following formula:

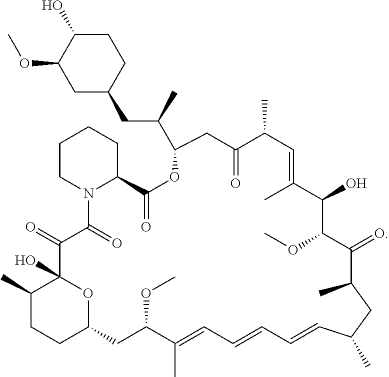

10. The pharmaceutical composition of claim 8, wherein the substituted quinoline is a chloroquine.

11. The pharmaceutical composition of claim 8, wherein the weight percentage ratio of the rapamycin to the substituted quinoline is between 1:100 and 1:5000.

12. The pharmaceutical composition of claim 8, wherein the rapamycin and the substituted quinoline cause a synergistic effect.

13. The pharmaceutical composition of claim 8, wherein the target therapy sensitizer is administrated together with the therapeutically active composition to improve or enhance the effect of the therapeutically active composition to a cancer or a tumor in an individual in need.

14. A target therapy sensitizer kit, comprising:
rapamycin and a first pharmaceutically acceptable carrier, diluent, excipient, or a combination thereof; and
substituted quinoline and a second pharmaceutically acceptable carrier, diluent, excipient, or a combination thereof.

15. The target therapy sensitizer kit of claim 14, wherein the rapamycin is a compound of the following formula:

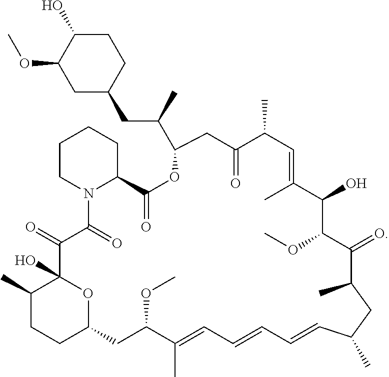

16. The target therapy sensitizer kit of claim 14, wherein the substituted quinoline is chloroquine.

17. The target therapy sensitizer kit of claim 14, wherein the rapamycin and the substituted quinoline cause a synergistic effect.

18. The target therapy sensitizer kit of claim 14, which is applied together with an effective amount of therapeutically active composition to an individual in need.

19. The target therapy sensitizer kit of claim 18, wherein the target therapy sensitizer kit utilizes the combination of rapamycin and substituted quinoline applied together with the therapeutically active composition to improve or enhance the effect of the therapeutically active composition to a cancer or a tumor in the individual in need.

20. A use of a combination of a rapamycin and a substituted quinoline as a target therapy sensitizer.

21. The use of claim 20, wherein the rapamycin is a compound of the following formula:

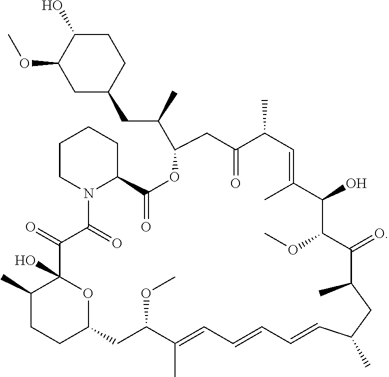

22. The use of claim 20, wherein the substituted quinoline is a chloroquine.

23. The use of claim 20, wherein the weight percentage ratio of the rapamycin to the substituted quinoline is between 1:100 and 1:5000.

24. The use of claim 20, wherein the rapamycin and the substituted quinoline cause a synergistic effect.

25. The use of claim 20, wherein the target therapy sensitizer is administrated together with an effective amount of therapeutically active composition to an individual in need.

26. The use of claim 25, wherein the target therapy sensitizer is administrated together with therapeutically active composition to improve or enhance the effect of the therapeutically active composition to a cancer or a tumor in the individual in need.

27. The use of claim 20, wherein the combination of the rapamycin and the substituted quinoline is administrated before, during or both before and during the target therapy.

28. The use of claim 20, wherein the target therapy sensitizer is used for treating a solid tumor.

29. The use of claim 20, wherein the target therapy sensitizer is a solid oral administration or a liquid oral administration.
